(19) United States
(12) Patent Application Publication
(10) Pub. No.: US 2017/0239304 A1
(43) Pub. Date: Aug. 24, 2017

(54) APPARATUS AND METHOD FOR EXTRACTING ESSENTIAL OIL

(71) Applicant: SHO PRODUCTS, LLC, NEW YORK, NY (US)

(72) Inventors: Vincent Terrell Pence, Frazier Park, CA (US); Samuel Jurist, New York, NY (US); Vincent D'Accolti, New York, NY (US)

(73) Assignee: SHO PRODUCTS, LLC, NEW YORK, NY (US)

(21) Appl. No.: 15/442,373

(22) Filed: Feb. 24, 2017

Related U.S. Application Data
(60) Provisional application No. 62/299,364, filed on Feb. 24, 2016.

(51) Int. Cl. A61K 36/185; B30B 15/34; B30B 9/20; A23D 7/005; A23D 7/04

(52) U.S. Cl. CPC A61K 36/185; A23D 7/005; A23D 7/04; B30B 9/20; B30B 15/34; A61K 2236/37; A61K 2236/53; A61K 2236/31

(57) ABSTRACT

An apparatus and system for extracting essential oil from plant material, and in particular, cannabis plant material, is described. The apparatus has top and bottom rollers that apply heat and pressure to the plant material as the plant material passes through the rollers for the extraction of essential oil. The top and bottom rollers are powered by a hand crank or motor, and the top and bottom rollers are heated by heaters regulated by a temperature controller. Also described is a method that uses a combination of heat, pressure, and speed for efficient extraction of high quality essential oil from plant material, and in particular, cannabis plant material.

### RELATED APPLICATION

The present application claims the benefit under 35 U.S.C. 119(e) of U.S. Provisional Appln. No. 62/299,364 filed Feb. 24, 2016, the entire contents of which are incorporated herein by reference.

### FIELD OF THE INVENTION

The present invention relates to a system, apparatus, and method for extracting essential oil from plant material, and in particular, for extracting essential oil from cannabis plants. The present invention is configured to extract essential oil from any strain of cannabis plant by applying heat and pressure.

### BACKGROUND OF THE INVENTION

Essential oils are known for having the characteristic fragrance of the plant from which they are derived such that they contain the “essence of” the plant's fragrance. Essential oils are typically used in perfumes, cosmetics, soaps, and household cleaning products. Essential oils have also been used for flavoring food and drink, and for medicinal purposes as well.

Interest in essential oils has peaked in recent decades with the popularity of aromatherapy which is a branch of alternative medicine that uses essential oils and other aromatic compounds for curative effects. When used for aromatherapy, essential oils are typically volatilized or diluted in a carrier oil and used in massage, diffused in the air by a nebulizer, heated over a candle flame, or burned as incense.

Extracting essential oil from cannabis plants is different from other types of plants because cannabis oil is more viscous and “sticky” in nature. Each strain of cannabis produces its own unique essential oil, and the viscosities of essential oils extracted from strains of cannabis range from viscous to almost solid. In view of the viscous nature of cannabis oil, there is a need for a system, apparatus, and method that properly extracts essential oil from cannabis plants.

### SUMMARY OF THE INVENTION

Methods for extracting essential oil from cannabis plants have typically involved the use of a solvent which is removed after the extraction of the essential oil is completed. A disadvantage of using solvents is that solvents are harmful to the natural properties of the essential oil. This is because methods that use solvents for extracting essential oil are extremely volatile processes that can destroy and/or separate many of the terpenes contained in the essential oil. Terpenes are important components of essential oils because they are responsible for the aroma and flavor characteristics of the essential oil.

Attempts to avoid the use of solvents for extracting essential oil from cannabis plants have used heated plates that are pressed against the plant material for extraction of the essential oil under heat and pressure. A problem with this method is that it must be done in small batches, and is thus time consuming. In order to increase production, larger plate sizes have been used. However, both the yield and the quality of the essential oil are compromised when scaling is done in this manner. This is because using larger plates increases the amount of plant material being pressed at once and this makes it difficult for the essential oil found in the center of the plant material to escape and make its way completely out of the plant material. Also, using larger heated plates causes the essential oil to sit on the heated plates for longer periods of time and this produces a lower quality essential oil because the terpenes in the essential oil will begin to degrade over time while the essential oil is in contact with the heated plate.

The present invention overcomes the aforementioned problems of cannabis oil extraction, in one example embodiment, by replacing the heated plates with a unique heated roller system that allows for a continuous feed of plant material for applying pressure and heat. The present invention in one aspect comprises among other features top and bottom rollers, a heating unit for heating the rollers, and a driving unit for driving the rollers. The top and bottom rollers apply heat and pressure to extract the essential oil from the cannabis plant. The present invention can thus enable a user to extract essential oil from the user's preferred strain of cannabis plant with ease and efficiency.

By virtue of the features of the present invention, the present invention can not only cut down on labor, it also can increase the quality of the essential oil extracted from the cannabis plant by minimizing the time the plant material is in contact with a heated surface. The present invention is also scalable for industrial production of large quantities of essential oil because there is no degradation of the quality of the essential oil when roller size is increased as the plant material will still only be in contact with a heat surface for a minimal amount of time no matter the size of the rollers.

The solution of the present invention to the problem of extracting essential oil from cannabis plants is unconventional because persons having ordinary skill in the art of cannabis oil extraction have had doubts as to the ability of a roller to sufficiently apply pressure and heat for the extraction of essential oil from cannabis plants. The inventors of the present invention were able to overcome the challenges of using a heated roller system by sourcing the appropriate type of material for the rollers and developing a novel way to heat the rollers in combination with determining an optimal pressure, temperature, and roller speed for extracting essential oils of high quality while simultaneously increasing production yield. Thus, the present invention is an unconventional solution with respect to previous methods and systems which use either solvents or heated plates, and which, for the reasons discussed above, produce essential oils of lower quality and lower yield because of the degradation of terpenes in the plant oil.

The present invention in a first aspect provides an apparatus for extracting essential oil from plant material comprising top and bottom rollers, means for rotating the top and bottom rollers, and means for adjusting the pressure applied by the top roller, and means for heating the top and bottom rollers.

The present invention in another aspect provides a method for extracting essential oil from plant material, comprising the steps of applying heat and pressure from top and bottom rollers to a plant material being fed through the top and bottom rollers, and wherein the top and bottom rollers are heated by a temperature controller.

The present invention in a third aspect provides a system for extracting essential oil from a cannabis plant, comprising top and bottom rollers, a first unit adapted to rotate the top and bottom rollers, and a second unit adapted to adjust the pressure applied by the top roller, wherein the top and bottom rollers are heated by a temperature controller.

### BRIEF DESCRIPTION OF THE DRAWINGS

FIG. 1 is a front view of a first embodiment of an apparatus according to the present invention for extracting essential oil from plant material.

FIG. 2 is a right side view of the first embodiment of the apparatus.

FIG. 3 is a left side view of the first embodiment of the apparatus.

FIG. 4 is a front perspective view of a second, preferred embodiment of the apparatus for extracting essential oils with the external housing removed.

FIG. 5 is a rear perspective view of the second embodiment of the apparatus with the external housing removed.

FIG. 6 is a perspective view of the second embodiment of the apparatus with the housing covering the internal components of the apparatus.

FIG. 7 is a front view of the second embodiment of the apparatus.

FIG. 8 is another front view of the second embodiment of the apparatus.

FIG. 9 is another front view of the second embodiment of the apparatus.

FIG. 10 is a view of a swivel electrical connector for heating a roller in the apparatus for both the first and second embodiments of the present invention.

FIG. 11 is a hollowed perspective view of the inside of each roller of the apparatus for both the first and second embodiments of the present invention.

FIG. 12 is another hollowed perspective view of the inside of each roller of the apparatus for both the first and second embodiments of the present invention.

### DETAILED DESCRIPTION OF THE INVENTION

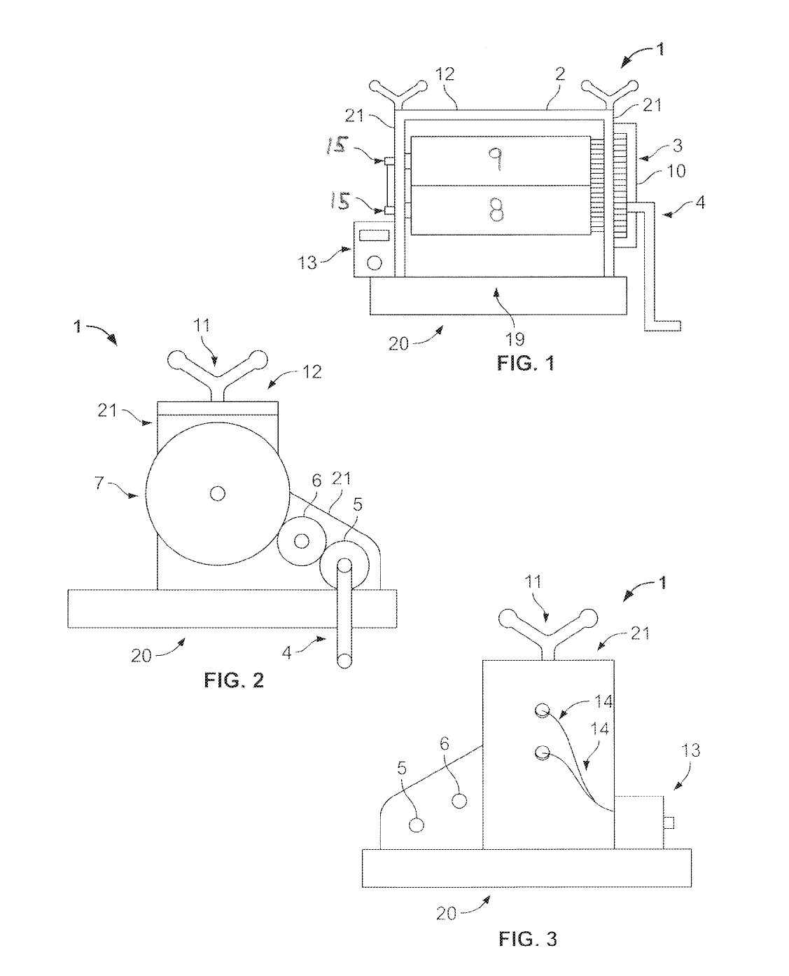

FIGS. 1, 2, and 3 show a first embodiment of the present invention which comprises an apparatus (1) for extracting essential oil from plant material. The apparatus (1) includes a housing (2) having a top plate (12), a pair of side plates (21), and a base (20). Inside the housing (2) is a main gear (5) capable of being turned by hand or motor power, for example. In the examples shown in FIGS. 1 and 2, a hand crank (4) is used for driving the main gear (5).

As shown in FIG. 2, the main gear (5) is engaged with a secondary gear (6) such that the main gear (5) drives the secondary gear (6). Furthermore, the secondary gear (6) is engaged with a large gear (7) such that the secondary gear (6) drives the large gear (7). The large gear (7) is engaged with a bottom roller (8) for rotating the bottom roller (8) as plant material is fed into the housing (2). Proper gear reduction from the main gear (5) to the large gear (7) is important in order to optimize the roller speed of the apparatus (1).

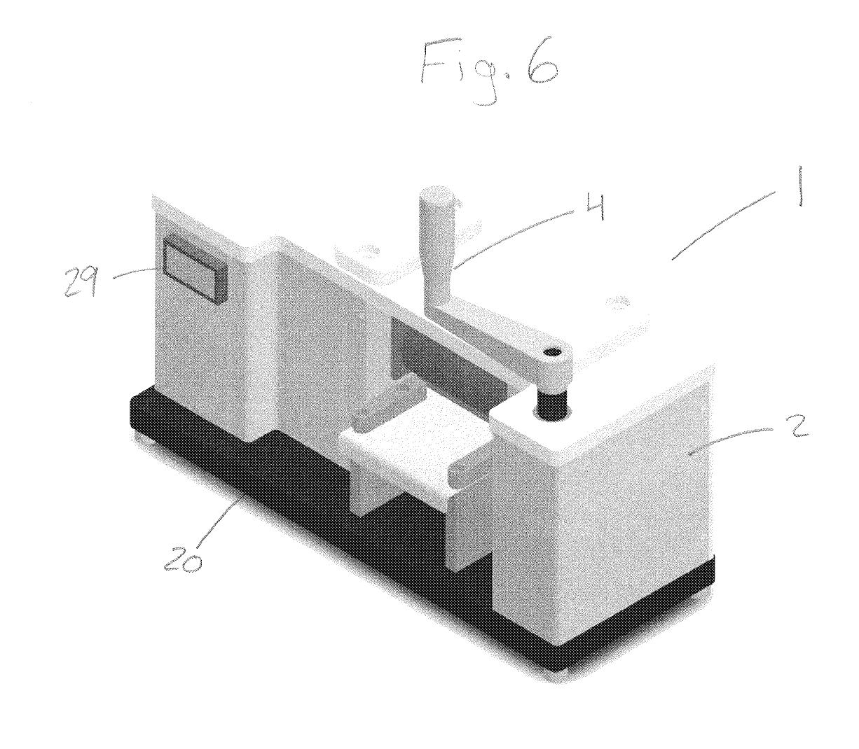

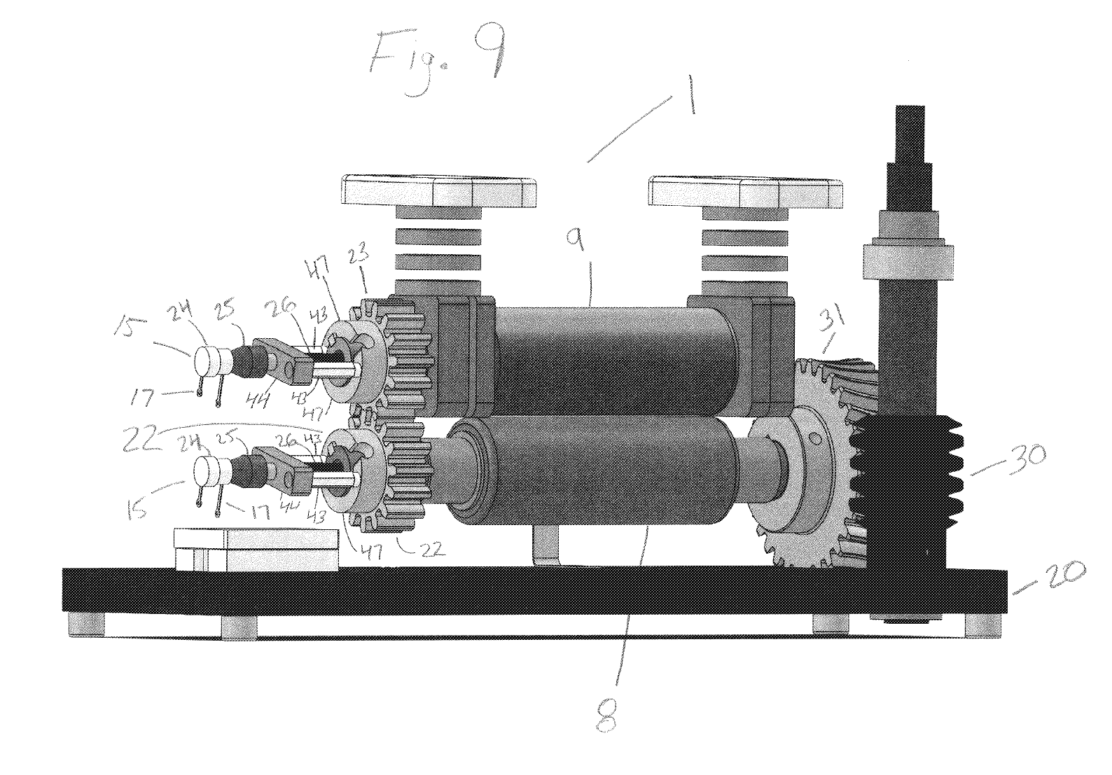

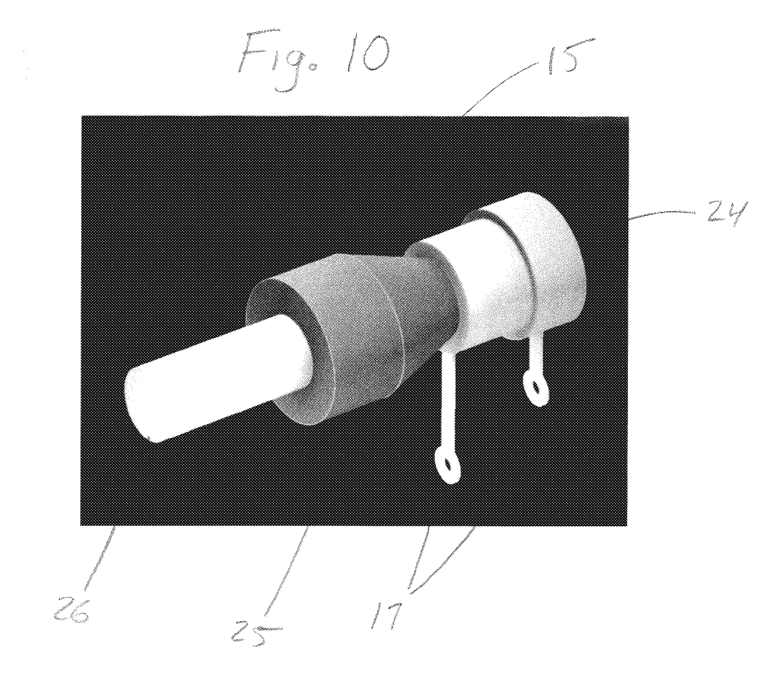

The apparatus (1) further comprises a top roller (9) so that the plant material is fed between the top roller (9) and the bottom roller (8). Both the top and bottom rollers (8, 9) rotate as the plant material passes through, and each roller applies pressure and heat on the plant material for extracting essential oil from the plant material. The bottom roller (8) on the opposite side of the main gear (5) comprises a first matching gear (22) which is engaged with a second matching gear (23) of the top roller (9). Although details of the first and second matching gears (22, 23) are not shown in FIGS. 1-3 of the first embodiment, the first and second matching gears (22) and (23) shown in FIGS. 4-9 of the second embodiment can be utilized, such as, for example, the matching gears (22) and (23) shown in FIGS. 8 and 9. Thus, when the bottom roller (8) rotates, it causes the top roller (9) to also rotate. The matching gears (22, 23) attached to the top and bottom rollers (8, 9) are modified by having a hole drilled into each of them for allowing swivel electrical connectors (15) to be properly mounted onto each roller for heating each of the rollers (8, 9) of the apparatus (1). Also, a reduction gear (3) can be used for slowing down the turning of the rollers (8, 9) to an acceptable speed so that heat and pressure can be properly utilized for extracting the essential oil from the plant material.

The top and bottom rollers (8, 9) are mounted in a channel (10) located in the housing (2) of the apparatus (1). The bottom roller (9) is fixed in position while top roller (8) is spring loaded in the channel (10) such that the pressure applied by the top roller (9) on the plant material that passes between the top and bottom rollers (8, 9) can be adjusted. Spindle bolts (11) located on the top plate (12) of the apparatus (1) are used for adjusting the pressure exerted on the plant material by the top roller (9) as the plant material passes through the rollers (8, 9).

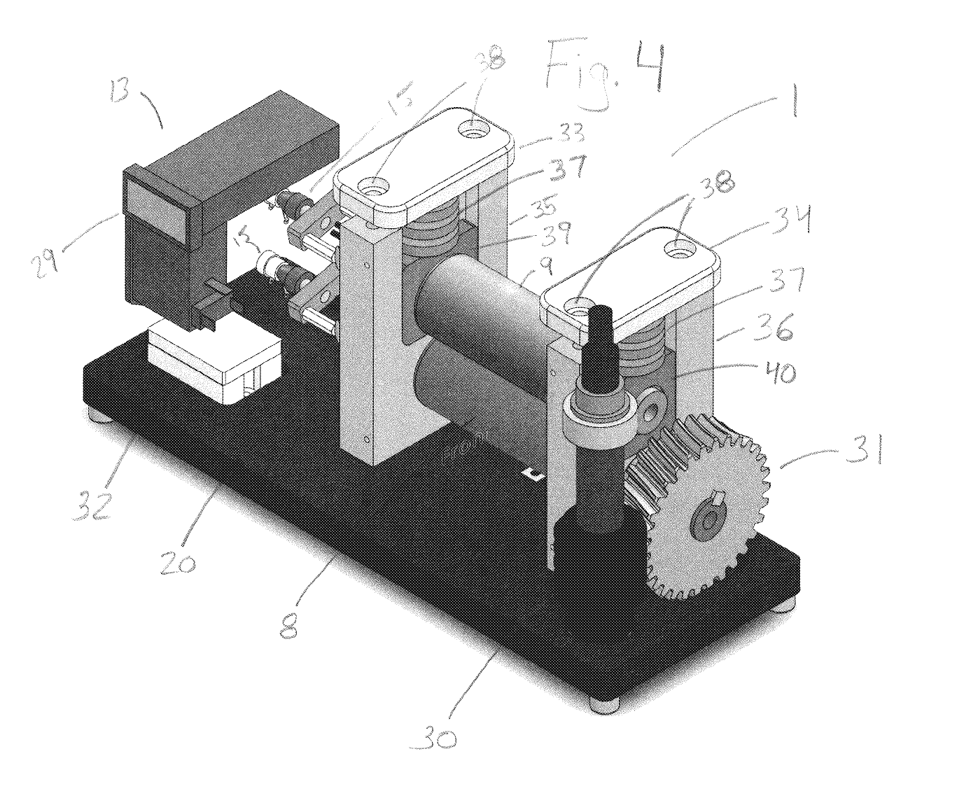

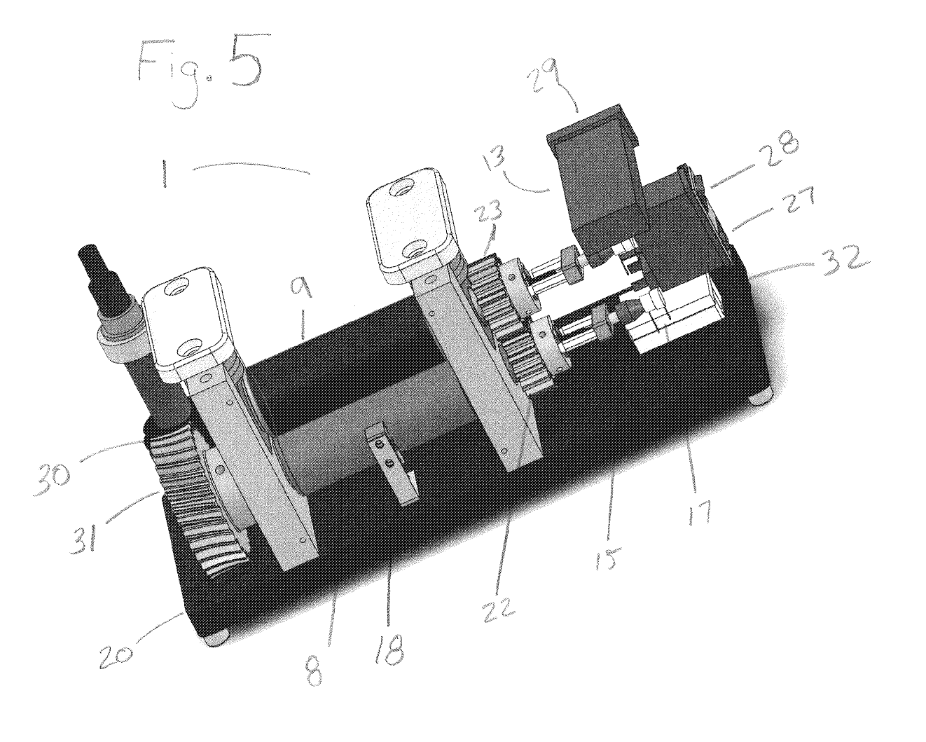

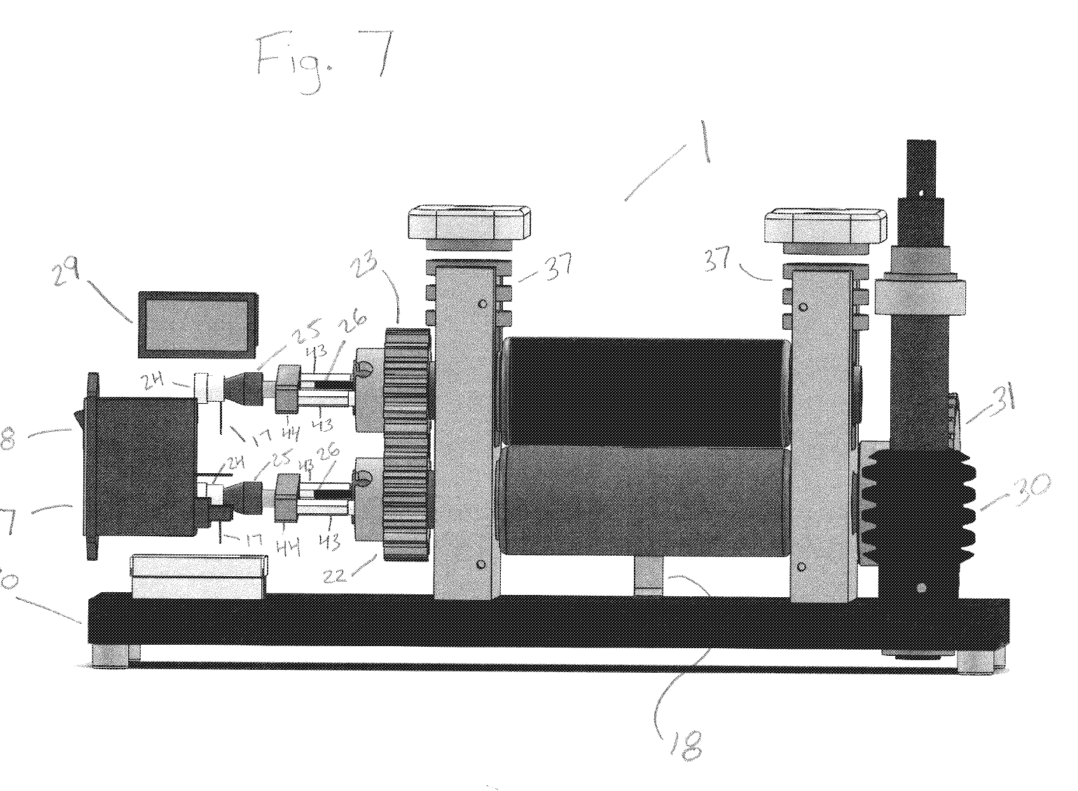

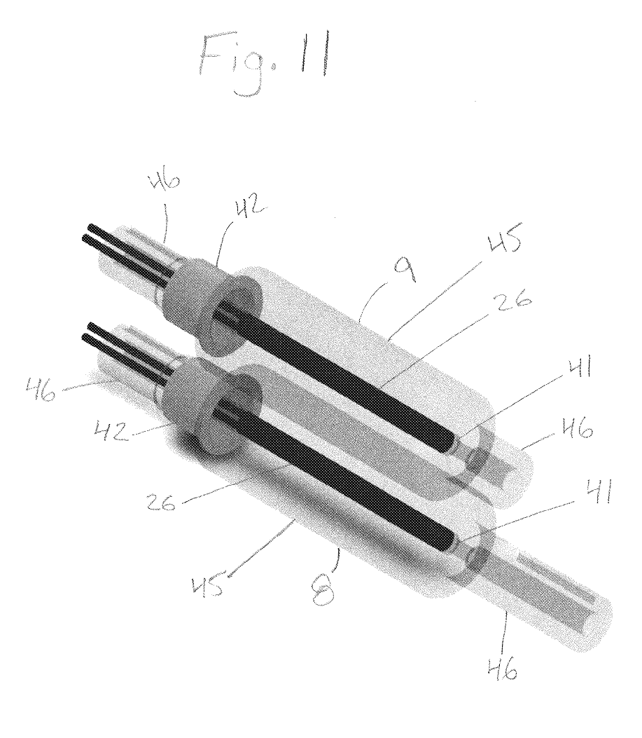

As shown in FIG. 3, the temperature controller (13) is mounted to the apparatus (1) with the insulated wires (14) running from the temperature controller (13) to the thermal couplings (17) of each swivel electrical connector (15) for each roller. Although details of the swivel electrical connectors (15) are not shown in FIGS. 1-3 of the first embodiment, the swivel electrical connectors (15) shown in FIGS. 4-9 of the second embodiment can be utilized, such as, for example, the swivel electrical connector (15) shown in FIG. 10. The temperature controller (13) comprises an electrical connector (27) for connecting the temperature controller to an electrical source such as a wall socket for supplying electricity through the insulated wires (14) to the swivel electrical connectors (15). The temperature controller (13) further comprises a switch (28) for turning on or off the temperature controller, and an interface (29) for a user to enter a desired temperature for heating the rollers (8, 9). The electrical connector (27), switch (28), and interface (29) shown in FIGS. 4 and 5 of the second embodiment can be used for the first embodiment of the present invention. Also, a relay (32) in the first embodiment of the present invention is connected to the temperature controller (13) as shown, for example, in the second embodiment of the present invention depicted by FIGS. 4 and 5.

FIGS. 4-9 depict a second, preferred embodiment of the present invention. In this embodiment, the hand crank (4) is located on the top of the apparatus as shown in FIG. 6. The hand crank (4) rotates about a vertical axis for rotating a cylindrical gear (30) about the vertical axis as shown in FIG. 4. The cylindrical gear (30) is engaged with a turning gear (31) that rotates about a horizontal axis and that is connected to the bottom roller (8) for rotating the bottom roller (8) about the horizontal axis. On the opposite side of the turning gear (31) as shown in FIGS. 8 and 9, the bottom roller (8) is connected to a first matching gear (22) which is engaged with a second matching gear (23) of the top roller. Thus, when the hand crank (4) is rotated about the vertical axis, the cylindrical gear (30) engages the turning gear (31) for rotating the bottom roller (8) about the horizontal axis, and the first matching gear (22) of the bottom roller engages the second matching gear (23) of the top roller for rotating the top roller (9). Thus, rotating the hand crank (4) about the vertical axis rotates both the top and bottom rollers (8, 9) about their respective horizontal axes. Like in the first embodiment, the matching gears (22, 23) attached to the top and bottom rollers (8, 9) in the second embodiment of the present invention are modified by having a hole drilled into each of them for allowing swivel electrical connectors (15) to be properly mounted onto each roller for heating each of the rollers (8, 9) of the apparatus (1).

FIG. 4 of the second embodiment shows a left top plate (33) and a right top plate (34) respectively mounted onto left and right support towers (35, 36). The left side of the bottom roller (8) is rotatably attached to the left support tower (35) and the right side of the bottom roller (8) is rotatably attached right support tower (36). The left side of the top roller (9) is rotatably attached to a left channel element (39) and the right side of the top roller (9) is rotatably attached to a right channel element (40). The left and right channel elements (39, 40) are slotted inside the left and right support towers (35, 36) respectively. A spring (37) is located in each of the left and right support towers (35, 36) and is compressed by the top plate (33, 34) in each of the support towers (35, 36). Screws (38) are used for connecting each top plate (33, 34) to each support tower (35, 36). By turning the screws (38) of each top plate (33, 34), the height of each top plate (33, 34) can be adjusted with respect to each support tower (35, 36). By adjusting the height of each top plate (33, 34), the pressure applied by the springs (37) in each support tower (35, 36) to the left and right sides of the top roller (9) is adjusted. Thus, the bottom roller (8) is fixed with respect to the vertical axis of the left and right towers (35, 36), and the pressure of the top roller (9) applied to the plant material passing through the top and bottom rollers (8, 9) can be adjusted by turning the screws (38) of the left and right top plates (33, 34) which changes the compression of the springs (37) applied to the left and right channel elements (39, 40). Since the top roller (9) is rotatably connected to the left and right channel elements (39, 40), the pressure applied to the plant material passing through the top and bottom rollers (8, 9) is adjusted by the change of the compression of the springs (37) in the support towers (35, 36).

The left and right top plates (33, 34) can be made, for example, from aluminum, whereas the left and right towers (35, 36) and the left and right moving elements (39, 40) can be made, for example, from stainless steel. Of course, it is also possible that these components be made from other types of suitable materials such as other types of non-corrosive metal alloys. The screws (38) for raising or lowering the left and right top plates (33, 34) and thereby compressing or decompressing the springs (37) in each of the left and right support towers (35, 36) can be of a hexagonal socket requiring an Allen key or hex key. It is also contemplated that instead of screws (38), threaded bolts and nuts can be used for raising or lowering the left and right top plates (33, 34). Also, FIGS. 4-9 show two screws (38) for each top plate (33, 34) are used in the second embodiment; however, it is contemplated that the number of screws for raising or lowering each top plate can vary such that one screw for each top plate or more than two screws for each top plate can be used for adjusting the height of each top plate (33, 34).

FIGS. 4 and 5 show the temperature controller (13) mounted to the apparatus (1). Like in the first embodiment, the temperature controller (13) comprises an electrical connector (27) for connecting the temperature controller (13) to an electrical source, a switch (28) for turning on and off the temperature controller (13), and an interface (29) for a user to enter a desired temperature for heating the rollers (8, 9). A relay (32) is connected to the temperature controller (13) as shown, for example, in FIGS. 4 and 5. Like in the first embodiment, the second embodiment of the present invention uses a swivel electrical connector (15) depicted in FIG. 10 for heating each roller of the apparatus (1). The swivel electrical connectors (15) comprise a stationary part (24), thermal couplings (17), a rotating part (25), and a heating element (26) that extends into each roller for heating the rollers (8, 9), as shown in FIGS. 4, 5, and 7-9.

FIG. 5 shows a back view of the second embodiment. In this figure, a ground (18) is shown for grounding the apparatus (1). The ground (18) can also be used for attaching the housing (2) to the base (20) of the apparatus (1).

FIG. 6 shows the second embodiment having the housing (2) attached to the base (20) thereby covering the internal components of the apparatus (1). FIG. 6 shows the hand crank (4) located on the top of the housing (2) and capable of being spun around a vertical axis. Also shown is the interface (29) for enabling a user to enter a desired temperature for heating the rollers (8, 9). The housing (2) can be made of stainless steel; however, it is also possible that the housing (2) can be made of other materials such as other types of non-corrosive metal or plastic.

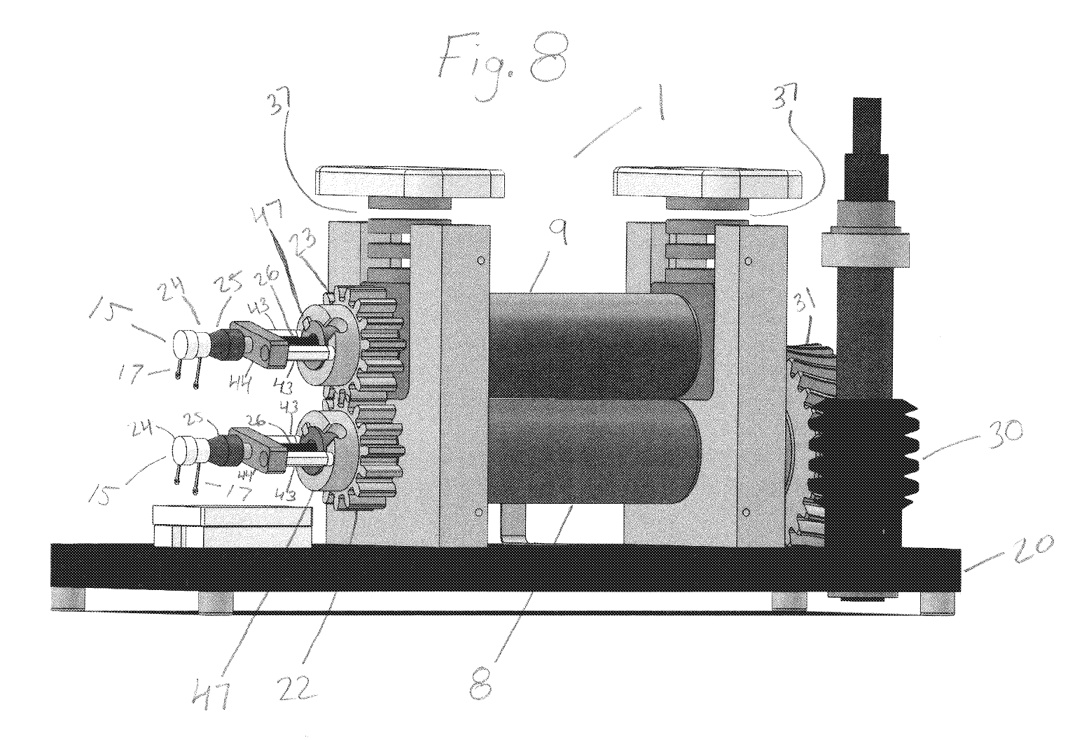

FIGS. 7-9 show additional views of the second embodiment of the present invention with the housing (2) removed and thereby showing the inner components of the apparatus (1). FIG. 7 is a front view of the apparatus (1) and shows from an eye-level point of view the top and bottom rollers (8, 9) through which the plant material passes through. FIGS. 8 and 9 similarly show the front of the apparatus (1) in the second embodiment from a left view perspective.

FIGS. 8 and 9 show ring insulators (44) for each of the swivel electrical connectors (15) connected to each of the rollers (8, 9). Each ring insulator (44) is a rectangular block which covers at least a portion of the outside surface of each heating element (26) that extends from each swivel electrical connector (15). The heating element (26) from each swivel electrical connector (15) extends through the center of each ring insulator (44), and each ring insulator (44) is connected by pairs of standoffs (43) to gaskets (47) fixed around left cylindrical extensions (46) of the top and bottom rollers (8, 9).

FIG. 10 shows an example of a swivel electrical connector (15) for heating each roller of the apparatus in both the first and second embodiments of the present invention. The swivel electrical connector (15) comprises a stationary part (24) having thermal couplings (17) that are connected to insulated wires (14) (shown in FIG. 3) running from the temperature controller (13). The swivel electrical connector (15) further comprises a rotating part (25) fixed around a cylindrical heating element (26). The rotating part (25) and heating element (26) are connected to each roller by the ring insulator (44) connected by a pair of standoffs (43) to the gasket (47) of each top and bottom roller (8, 9). See, for example, FIG. 8. This allows the rotating part (25) of each swivel electrical connector (15) to rotate with each roller. Each heating element (26) extends into each roller for heating the rollers (8, 9). Since both the top and bottom rollers (8, 9) are heated, the apparatus (1) comprises two swivel electrical connectors (15), one for each roller.

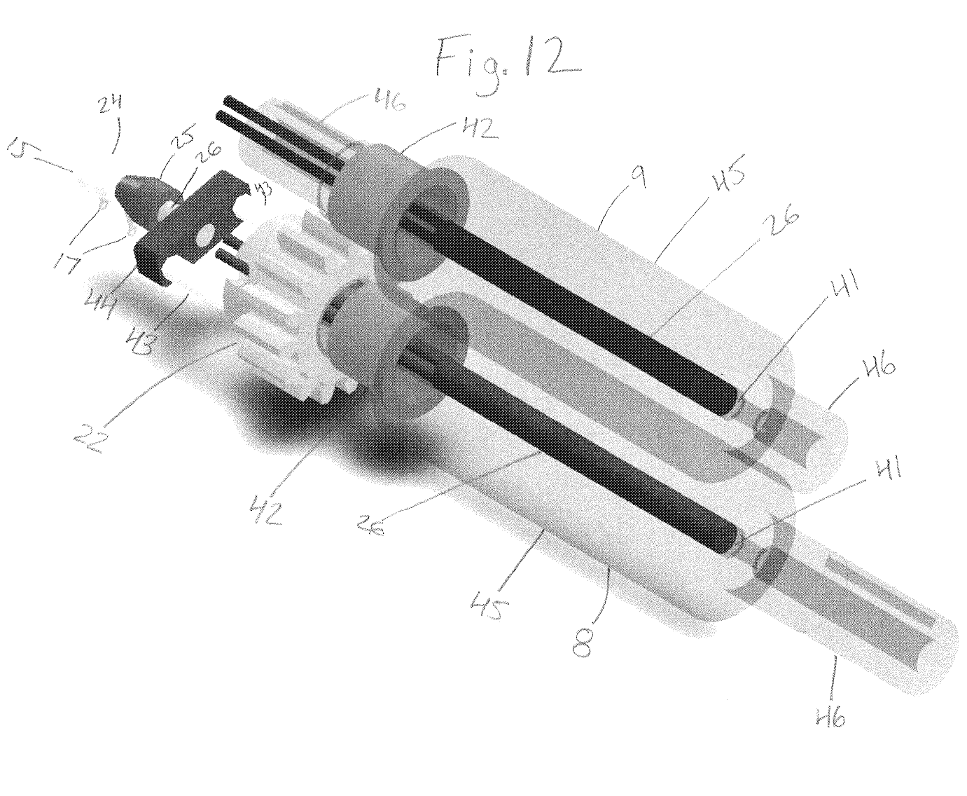

FIGS. 11 and 12 show hollowed views of the rollers (8, 9) of the apparatus (1) for both the first and second embodiments of the present invention. The heater elements (26) are located inside each roller. The top and bottom rollers (8, 9) are solid rollers preferably made from stainless steel. Each roller (8, 9) has a cylindrical shaped middle section (45) with cylindrical extensions (46) having a smaller diameter than the middle section (45) and extending from each end of the middle section (45). FIG. 11 shows inside each roller is a cylindrical bore (41) into which the cylindrical heating elements (26) from each of the swivel electrical connectors (15) are extended inside. An electrically non-conductive lubricant such as Watlube™ is used to fill the space between the cylindrical heater elements (26) and the contact surface of the cylindrical bores (41) of each roller and to thus improve the thermal conduction of heat from the heater elements (26) to the upper and lower rollers (8, 9). Bushings (42) are wrapped around each of the cylindrical extensions (46) of the rollers (8, 9) for insulating the rollers (8, 9) and preventing heat transfer from the rollers (8, 9) to the matching gears (22, 23) and to other parts of the apparatus (1) (it is noted that the bushing (42) are only shown on the left side cylindrical extension (46) of each roller (8, 9) in FIGS. 11 and 12, and that in the present invention the bushings (42) are also present on the right side cylindrical extension (46) of each roller (8, 9)).

FIG. 12 shows the same view of FIG. 11 except FIG. 12 also includes the first matching gear (22) of the bottom roller (8) and the swivel electrical connector (15) connected to the bottom roller (8). As stated above, the apparatus (1) comprises two swivel electrical connectors (15), one for each roller (and thus the second swivel electrical connector (15) connected to the top roller (9) is not shown in FIG. 12). A bushing (42) shown in FIG. 12 is fixed around the left cylindrical extension (46) of the bottom roller (8) thereby insulating the bottom roller (8) and preventing heat transfer from the bottom roller (8) to the matching gear (22). FIG. 12 further shows the swivel electrical connector (15) comprising the stationary part (24) having thermal couplings (17) that are connected to the insulated wires (14) that run from the temperature controller (13), and the rotating part (25) of the swivel electrical connector (15). As explained above, the rotating part (25) of each swivel electrical connector (15) has a cylindrical heating element (26) that extends into each roller for heating the rollers (8, 9).

In both the first and second embodiments of the present invention, a hand crank (4) is depicted for driving the gears of the apparatus (1) and rotating the top and bottom rollers (8, 9); however, it is contemplated that instead of, or in addition to the hand crank (4), a motor may also be used to drive the top and bottom rollers (8, 9). For example, an electrical motor can be used to power the top and bottom rollers (8, 9). The electrical motor can be powered, for example, by an external source of electricity or by an internal battery power source. In addition to electrical motors, other drive mechanisms can be used as well; for example, it is contemplated that any type of motor including internal combustion engines could be used to drive the top and bottom rollers (8, 9). It is also contemplated that the apparatus (1) can be equipped with both a motor and a hand crank (4) such that the hand crank (4) can be utilized in the event of a motor malfunction to power the top and bottom rollers (8, 9).

In both embodiments of the present invention, the surfaces of the top and bottom rollers (8, 9) are smooth and flat. However, it is also possible that the surfaces of the rollers (8, 9) are optionally embedded with grooves, bumps, and other geometric shapes to aid the compression of the plant material as it passes through the rollers (8,9). In a preferred embodiment, the top and bottom rollers (8, 9) are made from stainless steel; however, it is also possible that the rollers (8, 9) can be made from other materials such as other types of non-corrosive metal alloys.

When working with different types of plant material, adjusting the temperature and pressure applied by the apparatus (1) will aide in quality and yield of the essential oil extracted from the plant material. The top roller (8) in both embodiments is spring loaded by the spring (37). This allows the plant material to be fed into the top and bottom rollers (8, 9), and as the top and bottom rollers (8, 9) turn causing the plant material to pass through, the heat and pressure applied from the rollers (8, 9) to the plant material extracts essential oil from the plant material.

In both the first and second embodiments of the present invention, the temperature controller (13) is configured to regulate the top and bottom rollers (8, 9) to have a temperature of approximately 150° F. to 250° F., and more preferably, a temperature of approximately 180° F. to 200° F. for the extraction of essential oil from strains of cannabis plant. Temperatures above these ranges can degrade the terpenes in the cannabis essential oil thus diminishing the quality of the essential oil, and temperatures below these ranges may not effectively and/or efficiently extract the essential oil from the cannabis plant material.

In both the first and second embodiments of the present invention, the pressure applied by the spring biased top roller (9) onto the plant material that passes through the top and bottom rollers (8, 9) can be adjusted between approximately 300 psi and 4000 psi, and more preferably for strains of cannabis plants, the pressure can be adjusted between approximately 500 psi and 2000 psi. Pressures below this range may be insufficient for the efficient extraction of essential oil from the cannabis plant material and may thus lead to waste of cannabis plant material. Pressures above this range may cause blowouts leading the cannabis plant material to bust out of the filter bags, and may contaminate the rosin with the cannabis plant material. Also, pressures above this range may exert a force between the top and bottom rollers (8, 9) that can damage the apparatus (1) without a significant gain of oil extraction.

With respect to the speed of the rollers (8, 9), if the cannabis plant material is passed through the top and bottom rollers (8, 9) too quickly, it can result in a decrease in the yield of the essential oil extracted. Thus, the speed at which the top and bottom rollers (8, 9) rotate is critical for proper essential oil extraction from cannabis plants. As an example, in both the first and second embodiments of the present invention, the time the plant material is in contact with the rollers (8, 9) should be approximately 20 seconds to 5 minutes, and more preferably for strains of cannabis plants, approximately 30 seconds to 1 minute and 30 seconds. Faster speeds may reduce the yield of the essential oil extracted from the cannabis plant and thus lead to waste of cannabis plant material, however, faster speeds may also mean that the essential oil will spend less time under the heat and pressure applied by the heated rollers (8, 9) which improves the quality of the extracted essential oil. Slower speeds may increase the yield of the essential oil extracted from the cannabis plan material, however, slower speeds may also mean that the cannabis plant material will spend more time under the heat and pressure applied from the heated rollers (8, 9) which can lower the quality of the extracted essential oil.

The apparatus (1) in both embodiments of the present invention can be a small tabletop unit for personal and/or home use, or can be built on a larger scale for industrial production of essential oil, for example. In industrial operations, the apparatus (1) may be powered by a motor for feeding more plant material through larger rollers with larger heating elements.

The present invention in another aspect provides a method for extracting essential oil from plant material comprising the steps of using the apparatus (1) to apply heat and pressure from the top and bottom rollers to plant material being fed through the top and bottom rollers. Before being fed into the top and bottom rollers (8, 9), the plant material can be wrapped in a screen material and parchment paper. The plant material is placed in the screen material to allow the essential oil to escape while holding back the solid plant material. The parchment paper is used on the outside of filter bag to catch the extracted oil so that it can be gathered and collected. Thus, after passing through the top and bottom rollers (8, 9), the essential oil can be collected from the parchment paper. The screen material can be made of nylon mesh and can come in different micron ratings depending on the filtering and purity preferences of the user of the apparatus (1). Alternatively, the plant material can be fed into the rollers (8, 9) with only the screen material and no parchment paper. In this case, the essential oil drains off the bottom roller (8) into a tray (19) for collection. An advantage of the present invention over previous oil extractors and methods of plant oil extraction is that the oil extracted from the present invention flows in a single direction making its collection easier and less messy.

Any part of the plant that contains trichomes can be used for the extraction of essential oil. Trichomes are small hairs or other outgrowths from the epidermis of a plant. With respect to cannabis plants, trichomes are most commonly found in the flower and fan leaves of the plant. Thus, the system, apparatus, and method of the present invention typically use the flowers and fan leaves of the cannabis plant which are removed from the stem of the plant, and are pressed by the apparatus (1) for the extraction of essential oil from the cannabis plant.

The essential oils extracted from the system, apparatus, and method of the present invention can be used for smoking, cooking edibles, and topical creams. Also, because of the aromatic properties of essential oils, it is possible to use the essentials extracted from cannabis plants for perfumes, candles, incenses, soaps, and aromatherapy diffusers.

The present invention or certain part(s) or function(s) thereof, including, for example, the hand crank or power motor, the speed or temperature of the rollers, etc., may be controlled using hardware, software, or a combination thereof, and may be controlled by one or more computer systems or other processing systems. A computer system for controlling the operations of the present invention and capable of controlling the functionality described herein can include one or more processors connected to a communications infrastructure (e.g., a communications bus, a cross-over bar, or a network). Various software embodiments are described in terms of such an exemplary computer system. After reading this description, it will become apparent to a person of ordinary skill in the relevant art how to implement the present invention using other computer systems and/or architectures.

The computer system can include a display interface that forwards graphics, text, and other data from the communication infrastructure (or from a frame buffer) for display on a display unit. The display interface can communicate with a browser. The computer system also includes a main memory, preferably a random access memory, and may also include a secondary memory and a database. The secondary memory may include, for example, a hard disk drive and/or a removable storage drive, representing a floppy disk drive, a magnetic tape drive, an optical disk drive, etc. The removable storage drive reads from and/or writes to a removable storage unit in a well-known manner. The removable storage unit can represent a floppy disk, magnetic tape, optical disk, etc. which is read by and written to by the removable storage drive. As will be appreciated, the removable storage unit can include a computer usable storage medium having stored therein computer software and/or data.

The computer system may also include a communications interface which allows software and data to be transferred between the computer system and external devices. The terms “computer program medium” and “computer usable medium” are used to refer generally to media such as the removable storage drive, a hard disk installed in the hard disk drive, and signals. These computer program products provide software to the computer system.

Computer programs or control logic are stored in the main memory and/or the secondary memory. Computer programs may also be received via the communications interface. Such computer programs or control logic (software), when executed, cause the computer system or its processor to perform the features and functions of the present invention, as discussed herein.

Although the invention has been explained in relation to its preferred embodiments, it is understood that many other possible modifications and variations can be made without departing from the spirit and scope of the invention as herein described. Also, while the embodiments described above are generally described as relating to an apparatus, system, and method for extracting essential oil from cannabis plants, it is understood that the present invention may also be used for the extraction of essential oils from different types of plants and materials.

### LIST OF COMPONENTS OF THE INVENTION

1. Apparatus
2. Housing
3. Reduction gear
4. Hand crank
5. Main gear
6. Secondary gear
7. Large gear
8. Bottom roller
9. Top roller
10. Channel
11. Spindle bolts/roller adjustment
12. Top plate
13. Temperature controller
14. Insulated wire
15. Swivel electrical connectors
16. Hole drilled out heaters
17. Thermal couplings
18. Grounding
19. Optional tray
20. Base
21. Side plate
22. First matching gear
23. Second matching gear
24. Stationary part of the swivel electrical connector
25. Rotating part of the swivel electrical connector
26. Cylindrical heating elements
27. Electrical connector of the temperature controller
28. Switch of the temperature controller
29. Interface of the temperature controller
30. Cylindrical gear
31. Turning gear
32. Relay
33. Right top plate
34. Left top plate
35. Right support tower
36. Left support tower
37. Spring
38. Screw
39. Left channel element
40. Right channel element
41. Cylindrical bore inside each roller
42. Bushing
43. Standoffs
44. Ring insulator
45. Cylindrical middle section of each roller
46. Cylindrical extension of each roller
47. Gasket

1. An apparatus for extracting essential oil from plant material, comprising:
top and bottom rollers;
means for rotating the bottom roller;
means for adjusting pressure applied by the top roller to plant material that passes through the top and bottom rollers; and
means for heating the top and bottom rollers.

2. The apparatus of claim 1, further comprising a temperature controller for regulating the temperature of the top and bottom rollers.

3. The apparatus of claim 2, wherein the means for heating the top and bottom rollers is insulated wire running from the temperature controller to swivel electrical connectors attached to each of the top and bottom rollers.

4. The apparatus of claim 2, wherein the top and bottom rollers are regulated by the temperature controller to have a temperature of 150° F. to 250° F.

5. The apparatus of claim 1, wherein the means for rotating the top and bottom rollers is a hand crank.

6. The apparatus of claim 1, wherein the means for rotating the top and bottom rollers is an electric motor.

7. The apparatus of claim 1, wherein the means for adjusting the pressure applied by the top roller are compressed springs inside left and right support towers to which left and right sides of the top roller are respectively attached to.

8. The apparatus of claim 1, wherein the top roller is configured to apply a pressure of 500 psi to 2000 psi to plant material passing through the top and bottom rollers.

9. The apparatus of claim 1, further comprising a base having a tray for the collection of extracted essential oil from the plant material.

10. The apparatus of claim 1, wherein the essential oil is extracted from a cannabis plant.

11. A method for extracting essential oil from plant material, comprising the steps of:
applying heat and pressure from top and bottom rollers to a plant material being fed through the top and bottom rollers;
wherein the top and bottom rollers are heated by swivel electrical connectors.

12. The method of claim 11, wherein the plant material is a strain of cannabis plant.

13. The method of claim 11, wherein the heat applied to the plant material is 150° F. to 250° F.

14. The method of claim 11, wherein the pressure applied to the plant material is 500 psi to 2000 psi.

15. The method of claim 11, wherein the plant material is in contact with the top and bottom rollers for 30 seconds to 1 minute and 30 seconds.

16. The method of claim 11, wherein the plant material is wrapped in a screen material and parchment paper before being fed into the top and bottom rollers, and the essential oil is collected from the parchment paper after the plant material has passed through the rollers.

17. The method of claim 11, wherein the plant material is fed into the top and bottom rollers with a screen material, and the essential oil drains into a collection tray.

18. The method of claim 11, wherein the essential oil extracted from the plant material flows in a single direction.

19. A system for extracting essential oil from a cannabis plant, comprising:
top and bottom rollers;
a first unit adapted to rotate the top and bottom rollers; and
a second unit adapted to adjust the pressure applied by the top roller;
wherein the top and bottom rollers are heated by swivel electrical connectors.
